(19) United States
(12) Patent Application Publication
(10) Pub. No.: US 2023/0329567 A1
(43) Pub. Date: Oct. 19, 2023

(54) IMPLANTABLE BLOOD PRESSURE MONITOR

(71) Applicants: ECOLE POLYTECHNIQUE FÉDÉRALE DE LAUSANNE, Lausanne (CH); UTI LIMITED PARTNERSHIP, Calgary (CA); REGENTS OF THE UNIVERSITY OF MINNESOTA, Minneapolis, MN (US)

(72) Inventors: Grégoire COURTINE, Lausanne CHUV (CH); Aaron PHILLIPS, Calgary (CA); Jordan SQAIR, Lausanne CHUV (CH); David DARROW, Minneapolis, MN (US)

(21) Appl. No.: 18/057,164

(22) Filed: Nov. 18, 2022

Related U.S. Application Data
(63) Continuation of application No. PCT/CA2021/050688, filed on May 20, 2021.
(60) Provisional application No. 63/027,905, filed on May 20, 2020.

(51) Int. Cl. A61B 5/0215; A61B 5/00

(52) U.S. Cl. CPC A61B 5/02158; A61B 5/0031; A61B 2562/0247

(57) ABSTRACT

An implantable blood pressure monitor has two or more elements configured to wrap at least part way around a blood vessel. Changes in pressure within the blood vessel may be sensed by detecting movements of the elements or forces applied to the elements. A blood pressure monitor may be implanted without surgery. The blood pressure monitor may transmit real time blood pressure information to a receiver by wireless data transmission.

### CROSS-REFERENCE TO RELATED APPLICATION

This application claims priority from U.S. Provisional Patent Application No. 63/027,905 filed 20 May 2020 and entitled IMPLANTABLE BLOOD PRESSURE MONITOR which is hereby incorporated herein by reference for all purposes. For purposes of the United States of America, this application claims the benefit under 35 U.S.C. § 119 of U.S. Provisional Patent Application No. 63/027,905 filed 20 May 2020 and entitled IMPLANTABLE BLOOD PRESSURE MONITOR.

### FIELD

This invention relates to apparatus and methods for measuring blood pressure.

### BACKGROUND

There are a wide range of circumstances in which it is desirable to monitor a subject's blood pressure.

### SUMMARY

This invention has a number of aspects. These aspects may be practiced individually or in any combination and include, without limitation:
Apparatus for measuring blood pressure;
Methods for measuring blood pressure;
Methods for inserting implanted blood pressure measuring apparatus;
Methods for processing signals indicative of blood pressure;
Methods for powering an implanted blood pressure monitor; and
Apparatus for implanting blood pressure measurement devices.

An aspect of the invention relates to an apparatus for measuring blood pressure. The apparatus is an implantable monitoring device. The apparatus is configured to measure changes in pressure within a blood vessel of a subject so as to obtain blood pressure measurements of the subject. The apparatus may transmit real time blood pressure measurements to a receiver by wireless data transmission.

In some embodiments, the apparatus comprises a first body dimensioned to sit adjacent to an outer wall of a blood vessel, an element connected to the first body dimensioned to extend at least partly around the other wall of the blood vessel, and one or more sensors operative to detect a force applied on the element and/or displacement of the element relative to the first body resulting from pulsations of the blood vessel. A module may be communicatively connected to the one or more sensors. The module may be operative to process signals detected by the one or more sensors and to output a blood pressure measurement.

In some embodiments, the first body comprises a hub with a first face and a second opposing face. In some embodiments, the element comprises a pair of arms, a first arm and a second arm, extending transversely from the first body. The first and second arms may comprise a radius of curvature that is equal to a distance between the hub and a center of the blood vessel. The first and second arms may be symmetrical. The arms may span around the blood vessel at an angle between about 160 to about 180 degrees. The one or more sensors may be arranged on one or both of the first and second arms.

In some embodiments, the arms are retractable into a cavity within the hub. The hub may comprise first and second openings at its opposite sides to allow the arms to extend through each of the openings from within the cavity. An actuation system may be arranged within the hub operable to move the arms between an extended and retracted position. There are different ways to store the arms in the retracted position within the hub. For example, a spindle may be arranged within the hub. In such example embodiment, the hub includes first and second slits to allow the arms to extend through from within the cavity as the arms are being rolled out of the spindle. The angle between the first and second slits relative to the center of the spindle may for example be less than about 180 degrees. In other embodiments, the first and second arms may be pivotally mounted to the hub. The first and second arms may form a dihedral angle when the arms are in its extended position.

The first and second arms have properties and characteristics suitable for use in a subject's body. The first and/or second arms may comprise rounded tips and/or edges. The arms may be made of a flexible and/or biocompatible material. A coating layer (e.g., one or more of a slippery coating, lubricant coating, and biocompatible material) may be bonded on surfaces of the first and/or second arms.

In some embodiments, the element comprises a second body. The second body is connectable to the first body by the one or more sensors. The sensors may be displaceable. In some embodiments, the first and second bodies have the same dimensions (e.g., width and length).

The one or more sensors that may be used with the apparatus include strain gauges, pressure sensors, force sensors, distance sensors (e.g., capacitive sensors, electrical resistance sensors, electrical impedance sensors, ultrasonic sensors, light sensors, and magnetic field sensors), angular position sensors, near infrared spectroscopy (NIRS) sensors, ultrasound sensors, and blood chemistry sensors.

The first and/or second body may each have a first face and second opposing face. The first face may comprise a concave surface. The radius of curvature of the first face may be in the range of from 1.5 to 2 mm. The first face may subtend an arc in the range of from about 50 degrees to about 60 degrees around a center of curvature of the first face. The second face may comprise a convex surface. In some embodiments, the first and/or second body has a thickness of less than 3 mm, or less than 2 mm. The first and/or second body may comprise a sharp, distal end along its longitudinal axis.

In some embodiments, the first and/or second body also includes other features. Such other features include for example one or more of a camera, an ultrasound transducer, an infrared source and detector, an ultrasound reflector, an antenna operatively connected to an oscillator or transmitter, and sensors.

In some embodiments, the module is communicatively connected to a receiver, operative to transmit the blood pressure measurement to the receiver (e.g., a watch, a module on a necklace, an adhesive patch, a wrist band, a tablet and a phone). The module may include one or more of a power supply, data processor, and transmitter or receiver. The module may comprise a housing with a diameter of less than 3 cm, and/or a length of less than 1 cm, or less than 6 mm.

The apparatus may include tools to assist with the delivery of the first body and the element into the subject. In some embodiments, the apparatus also includes an introducer tool, a probe extending from the introducer tool, and a coupling releasably connecting an end of the probe to the first body. The introducer may include an actuating mechanism operative to remotely control a movement of the first and second arms between the extended position and the retracted position. The actuating mechanism may also be operative to disconnect the coupling from the probe.

An aspect of the invention relates to a method of implanting a blood pressure monitor. The method involves delivering the blood pressure monitor comprising a first body and an element connected to the first body to a target location in a subject, orienting the first body at the target location which is adjacent to an outer wall of a blood vessel, and orienting the element to extend at least partly around a circumference of the outer wall of the blood vessel.

In some embodiments, an introducer carrying the first body and the element is used to deliver the blood pressure monitor to the target location. Such method involves orienting the introducer on a path intersecting the blood vessel at an angle, positioning the first body and the element on the path at the target location prior to delivering the blood pressure monitor to the target location, and advancing the introducer through a tissue of the subject along the path at the target location. In some embodiments, the first body is attached to the introducer after advancing the introducer through the tissue of the subject.

In embodiments in which the element comprises first and second extendable arms, the method may involve actuating the introducer to extend the arms from within a cavity of the first body after orienting the first body at the blood vessel.

In some embodiments, a probe is secured to the introducer at a first end and to the first body at a second opposing end. The method may include disconnecting the first body from the second opposing end of the probe after orienting the element at the blood vessel.

In embodiments in which the blood pressure monitor also includes a module that is operatively connected to the first body, the method includes delivering the module via the introducer to a second location spaced from the target position. In some embodiments, the step of advancing the module is performed before the step of orienting the first body at the blood vessel. The introducer may be actuated to extend the arms from within the cavity of the first body after the step of delivering a module to the second location.

In some embodiments, the method involves retracting the introducer after the step of delivering the module to the second location so as to separate the first body from the module and to deliver the first body to the target location.

The method may also involve removing the blood pressure monitor from the subject by incising a tissue of the subject near the target location. The first and second arms may be retracted into the first body before removing the first body through the incision.

An aspect of the invention relates to a method of measuring blood pressure. The method involves implanting a blood pressure monitor, orienting the first body adjacent to an outer wall of a blood vessel, orienting the element to extend at least partly around a circumference of the outer wall of the blood vessel, and detecting at one or more sensors a force applied on the element and/or displacement of the element relative to the first body resulting from pulsations of the blood vessel. The method may further involve transmitting the signals from the one or more sensors to a processor, and processing signals at the processor detected at the one or more sensors to obtain blood pressure measurements. The processor may be arranged inside or outside of a body of the subject. In some embodiments, the blood pressure measurements may be processed to determine one or more of systolic blood pressure (SBP), diastolic blood pressure (DBP) and mean arterial pressure (MAP). The processor may be calibrated by comparing signals received from the one or more sensors with blood pressure measurements generated from a second system.

Further aspects and example embodiments are illustrated in the accompanying drawings and/or described in the following description.

It is emphasized that the invention relates to all combinations of the features described herein, even if these are recited in different claims.

### BRIEF DESCRIPTION OF THE DRAWINGS

The accompanying drawings illustrate non-limiting example embodiments of the invention.

FIG. 1 is a schematic illustration showing an implantable blood pressure monitor according to an example embodiment.

FIG. 2 is a schematic view illustrating placement of an implanted blood pressure monitor relative to a blood vessel.

FIG. 2A is a schematic illustration showing an implantable blood pressure monitor according to a second example embodiment.

FIG. 3 is a schematic view showing an insertion tool coupled to an implantable blood pressure monitor.

FIGS. 4A, 4B, 4C, 4D and 4E are schematic illustrations showing different approaches to providing extendable arms.

FIGS. 5A and 5B are side elevation views that schematically illustrate an implantable blood pressure monitor being implanted.

### DETAILED DESCRIPTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive sense.

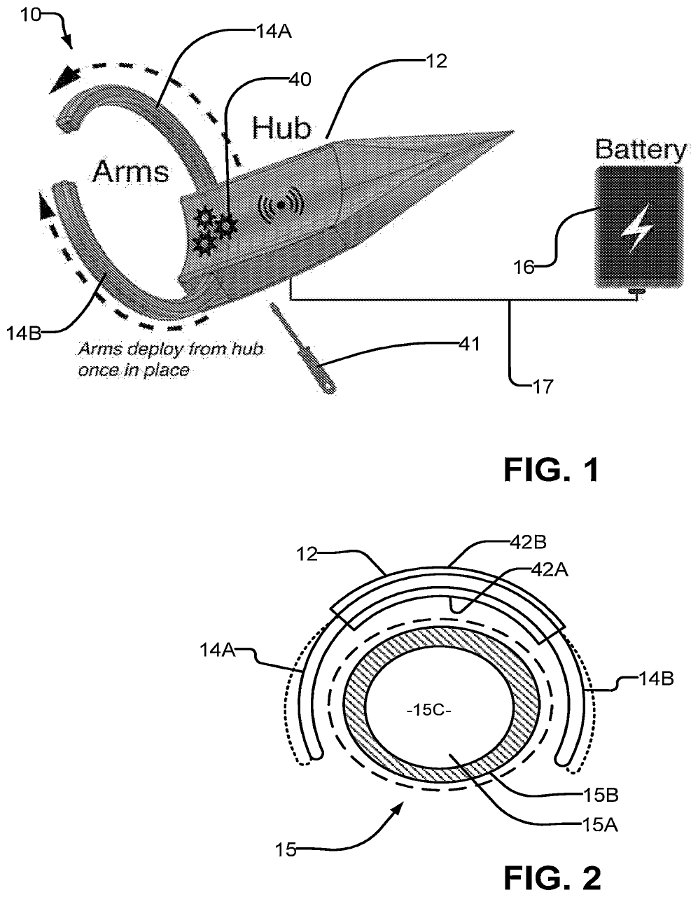

FIG. 1 shows an example implantable monitoring device 10. Device 10 comprises a hub 12 which supports at least one arm 14. The embodiment of FIG. 1 includes first and second arms 14A and 14B. One function of device 10 is to monitor blood pressure of a person or animal. In device 10, blood pressure monitoring is achieved by sensing forces on arm(s) 14 and/or monitoring displacements of one or more of arms 14. Arms 14 may, for example, comprise a relatively soft flexible biocompatible plastic material with a center reinforcement. The center reinforcement may help to ensure that arms 14 will not break while embedded in the subject.

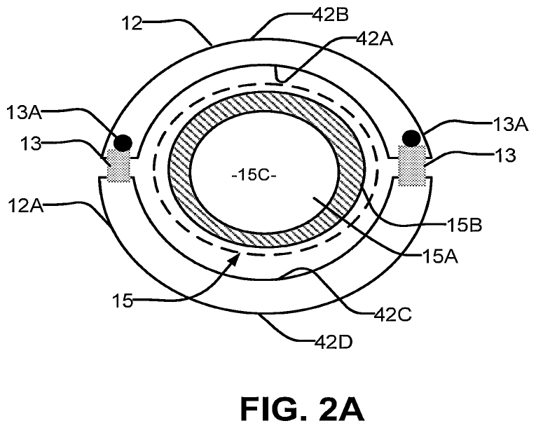

FIG. 2 illustrates hub 12 implanted adjacent to a blood vessel 15 having a lumen 15A and a wall 15B that contains blood 15C within vessel 15. Hub 12 is implanted either in or outwardly adjacent to wall 15B.

The pressure in blood 15C changes in with the phase of the subject's cardiac cycle. A lowest blood pressure (diastolic pressure) corresponds to diastole, the phase of the cardiac cycle where the subject's heart muscle relaxes. A highest pressure (systolic) corresponds to systole, the part of the cardiac cycle where the subject's heart muscle contracts to expel blood from the heart into the subject's arteries. In response to these changes in the pressure within lumen 15B vessel 15 expands and contracts in the radial direction.

Since hub 12 and arms 14 are located adjacent to vessel 15, the expansion and contraction of vessel 15 (the expanded configuration of vessel 15 is schematically indicated by dashed lines in FIG. 2) applies forces to arms 14 and/or causes arms 14 to move relative to one another and/or relative to hub 12. The magnitude of these forces and/or movements can be correlated to the value of the subject's blood pressure. The dotted lines in FIG. 2 schematically indicate relative displacement of arms 14 as a result of pulsation of vessel 15. By monitoring such forces and/or motions one can obtain continuous real time measurements of the subject's blood pressure.

When device 10 is in use, arm(s) 14, extend at least part of the way around the circumference of vessel 15. In the illustrated embodiment, arms 14 are curved so that they extend from hub 12 to at least roughly follow a circle centered on vessel 15. For example arms 14 may each be curved with a radius of curvature that is approximately equal to a distance between hub 12 and a center of vessel 15. Arms 14 may provide tension around vessel 15 without occluding vessel 15.

In some embodiments, arms 14 span an angle relative to a centerline of vessel 15 that is at least 160 degrees or at least 180 degrees. When arms 14 span an angle from the outer tip of arm 14A to the outer tip of arm 14B that extends at least approximately half way around vessel 15 then the pulsation of vessel 15 forces the tips of arms 14 apart when the blood pressure in vessel 15 at the location of hub 12 is increasing and allows the tips or arms 14 to move together when the blood pressure within vessel 15 at the location of hub 12 is decreasing.

Preferably, with arms 14A and 14B extended, device 10 spans an angle around vessel 15 that is more than 180 degrees. In such cases device 10 is held in place adjacent to vessel 15 by arms 14A and 14B wrapping more than half way around the vessel 15.

The forces on arms 14 and/or displacements of arms 14 which occur as a result of changing pressure within vessel 15 may be measured in any of a variety of ways including, for example, by way of:
strain gauges on arms 14 and arranged to detect bending of arms 14 in response to the pulsation of vessel 14 and/or
force sensors (e.g. piezoelectric force sensors) arranged to measure forces between two arms 14 and/or between arms 14 and hub 12 and/or between arms 14 and tissues adjacent to arms 14 and/or
sensors that directly or indirectly monitor changes in distance between two arms 14 or between points on arms 14 and hub 12. Such sensors may operate according to any suitable principle. For example, such sensors may comprise one or more:
   capacitive sensors;
   sensors that measure electrical resistance or impedance between electrodes on different ones of arms 14;
   sensors that measure time of flight between an ultrasound transmitter on one arm 14 and an ultrasound receiver on an opposing arm 14;
   sensors that measure transmission of light (e.g. infrared light) from a light source on one arm 14 to a light detector on an opposing arm 14;
   sensors that measure a magnetic field from a magnet on one arm 14 at a location on a second arm 14; and/or
   sensors operative to measure changes in angle at one or more joints in arms 14 or joints connecting arms 14 to hub 12.

To obtain accurate direct blood pressure measurements it is generally desirable to use a sensing modality which correlates as directly as possible to instantaneous blood pressure in vessel 15. For example, one or more sensors which directly measure forces on arms 14 and/or movements of arms 14 as a result of pulsation of vessel 15 are used in some example embodiments.

Vessel 15 may be any suitable blood vessel. Device 10 may be dimensioned to fit around the blood vessel. Devices 10 may be supplied in a range of different sizes to accommodate different blood vessels, different sites and/or differences in the anatomies of different subjects. A physician may select a site for device 10 that meets the needs of an individual subject.

In some embodiments device 10 is dimensioned to measure blood pressure in a small or medium-sized blood vessel such as the radial, ulnar, popliteal tibial artery. This does not mean that other embodiments in which apparatus 10 is dimensioned to be used to measure blood pressure in larger arteries such as the brachial artery, aorta or femoral artery.

In an example embodiment apparatus 12 is dimensioned to measure blood pressure in a small vessel. For example, vessel 15 may be a small peripheral artery such as the radial artery. The radial artery in adult humans typically has a diameter of about 3 mm. The wall of the radial artery is typically about 0.2 mm to 0.5 mm thick.

Hub 12 and arms 14 may be sized to match the size of vessel 15. For example, arms 14 may have lengths to wrap sufficiently around vessel 15. In some embodiments, arms 14 have a length equal to or less than a length or width of hub 12. Arms 14 may be fixed in position relative to hub 12, or adjustable in position by manipulation of e.g., a fastener. Where vessel 15 is the radial artery in an adult human, arms 14 may be configured to wrap at least part way around a 3 mm diameter cylinder. Hub 12 may be made small so that it can be implanted and remain implanted for a period of time without disturbing the subject. For example, hub 12 may have a thickness that is 3 mm or less, and is preferably 2 mm or less.

Device 10 may include electronics and/or optoelectronics that acquire measurements (e.g. outputs from one or more sensors that detect forces on and/or motions of arms 14) and transmit those measurements to another location which may be outside of the subject's body. For example, the measurements may be encoded into an optical or electromagnetic signal and transmitted from device 10 to a receiver located outside of the subject or to another system implanted in the subject.

Some processing may be performed on the measurements before the measurements are transmitted. For example, the measurements may be filtered before they are transmitted. For example, device 10 may include a high pass filter that eliminates lower frequency signals that may arise due to muscle motions. The measurements are optionally digitized (e.g. by an analog to digital converter that is part of device 10).

Any of a wide range of protocols may be used to transmit the measurements. For example a Bluetooth™ protocol, near field communication (NFC), WiFi, wireless infrared, or other wireless data protocol may be used to transmit measurement data from device 10 to outside of the subject's body and/or to another implanted system.

The receiver may optionally be in an object worn by the subject (e.g. a watch, a module on a necklace, an adhesive patch, a wrist band or the like). Alternatively, the receiver may be in an object often carried by the subject (e.g. a phone, tablet, wearable computing device or the like). The receiver may include circuits (which may optionally include a programmed data processor) configured to process the received measurements and, if necessary, perform additional processing to obtain one or more output values which may include calibrated blood pressure measurements for the subject in real time. The date and/or time on which each of the measurements are taken may be stored in memory.

In some embodiments, blood pressure measurements from an implanted device 10 provide feedback in a closed loop system for delivering a therapy. For example, blood pressure signals may be used as feedback to control a device that delivers a drug and/or a stimulation signal to the subject to alter the subject's blood pressure. Such closed loop systems may, for example, be applied in the control of hypertension or hypotension.

Device 10 may be powered by an implanted battery and/or an implanted power generator (that for example converts motions of arms 14 into electrical power) and/or a transcutaneous wireless power transmission system (e.g. a system that transmits electrical power to device 10 by inductive and/or capacitive coupling of an electromagnetic signal from an external antenna to an antenna implanted in the subject).

In some embodiments, device 10 includes a module 16 that is connected to hub 12 by a cable 17. Module 16 may, for example, comprise components for powering device 10 and/or processing signals detected by sensors of device 10 and/or transmitting signals to an external antenna and/or receiving electrical power transcutaneously. Providing some functional components of device 10 in separate module 16 facilitates making hub 12 small. In some embodiments module 16 has the form factor of a small cylinder. For example, module 16 may comprise a cylindrical housing about 3 mm or less in diameter. Module 16 may, for example, have a length of 1 cm or less, preferably 6 mm or less.

In some embodiments device 10 is fabricated for extended operation while implanted in a subject (e.g. over weeks, months, or years). To facilitate long term operation device 10 may include features such as:
a biocompatible surface coating. In some embodiments the surface coating may resist attachment to surrounding tissues. In some embodiments the surface coating comprises Teflon; and
a long-life, rechargeable and/or continuously charged electrical power supply.

Device 10 may optionally be implanted by surgery (e.g. creating an incision to expose a location at which it is desirable to implant hub 12—and module 16 if applicable—and then closing the incision). In some embodiments, however, device 10 is configured to allow device 10 to be implanted without surgery. Such embodiments can be advantageous, for example because:
they may expose the subject to less trauma,
they may be less expensive to implant;
the range of medical personnel qualified to non-surgically implant a device 10 is generally broader than the range of medical personnel qualified to surgically implant a device 10; and/or
non-surgical implantation can be safely performed under a wider range of conditions than surgical implantation.

For non-surgical implantation, hub 12 may include one or more of the following adaptations:
Hub 12 may be configured to attach to an introducer tool that may be used to insert hub 12 at a desired position and orientation relative to a vessel 15. The introducer tool may, for example, be needle-like. The introducer tool may allow hub to be implanted using a procedure much like intravenous cannulation except that hub 12 is preferably placed adjacent to and outside of the lumen of vessel 15 and does not penetrate vessel 15.
Arms 14 may be retractable into hub 12 and may be extended from hub 12 once hub 12 is in a desired position and orientation relative to a vessel 15. Hub 12 may include a mechanism for extending arms 14.

In some embodiments (e.g. the FIG. 2 embodiment and other embodiments) hub 12 comprises a flattened body that is relatively thin in a first transverse direction and is significantly wider in a second transverse direction. A hub 12 that has this shape may be oriented with a first large face 42A facing toward a vessel 15 and a second opposing large face 42B facing away from the vessel 15. In some embodiments the hub 12 subtends an arc of at least 50 degrees or at least 60 degrees around a center of curvature of first face 42A.

In some embodiments the first large face is concave. For example, the first large face may have a radius of curvature in the range of about 1.5 to 2 mm to facilitate receiving a vessel 15 having a diameter of about 3 mm such that the first large face wraps part way around outside of the vessel 15.

In FIG. 2 and some other example embodiments, hub 12 has a concave face 42A on a side intended to face a vessel 15 and openings 37A and 37B are oriented so that arms 14A and 14B leave hub 12 following a circumference of a circle of a radius that is selected to pass around a desired vessel 15. Arms 14A and 14B may be formed to have a curved shape with the same radius of curvature. A face 42B of hub 12 opposed to concave face 42A may optionally be convex. Other embodiments may have hubs 12 that share some or all of these features of configuration.

In some embodiments (e.g. the FIG. 2A embodiment), first and second arms 14A, 14B are replaced by a second hub 12A. Second hub 12A may have the same or different dimensions (e.g., length, width, thickness) and shapes as first hub 12. Similar to first and second faces 42A, 42B of first hub 12, second hub 12A may comprise a first and a second opposing face 42C, 42D. First opposing face 42C may be arranged to face vessel 15, and second opposing face 42D may be arranged to face away from vessel 15. First hub 12 may be arranged to extend a first portion of the circumference of vessel 15, and second hub 12A may be arranged to extend a second portion of the circumference of vessel 15. The combined transverse widths of first and second hubs 12, 12A allow hubs 12, 12A to wrap sufficiently around vessel 15. In some embodiments, first and second hubs 12, 12A may be oriented opposite to each other around vessel 15.

First and second hubs 12 and 12A may be connected by extensible elements 13 that allow first and second hubs to move toward and away one another as vessel 15 expands and contracts with cardiac pulses. Extensible elements 13 may, for example, comprise elastically extensible cords or sutures, elastically extendable strips or springs, mechanical linkages, etc.

Sensors 13A associated with first hub 12, second hub 12A and/or extensible elements 13 may operate to monitor one or more of:
relative displacement of first hub 12 and second hub 12A,
force exerted by vessel 15 on first hub 12 and/or second hub 12A;
a degree of extension of extensible elements 13;
tension in extensible elements 13;
etc.

In some embodiments, one or more sensors 13A is connectable to first and/or second hubs 12, 12A. One or more sensors 13A may be displaceable. One or more sensors 13 may be operative to measure forces applied on and/or between first and/or second hubs 12, 12A and/or displacement of first and/or second hubs 12, 12A relative to one another which occur as a result of changing pressure within vessel 15.

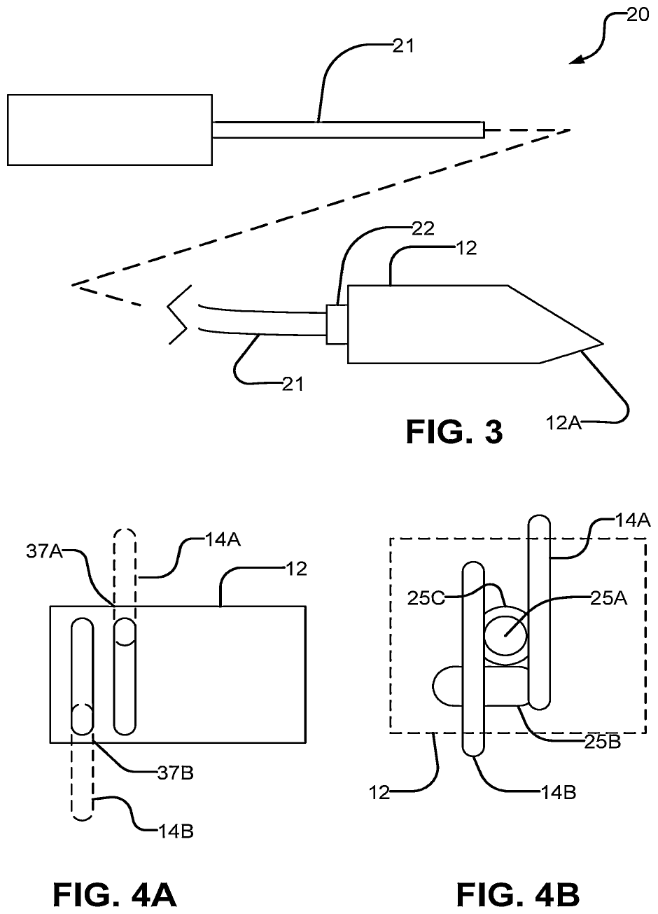

FIG. 3 schematically illustrates an example introducer tool 20 connected to hub 12. Introducer tool 20 includes a needle-like probe 21. A coupling 22 at an end of probe 21 attaches to hub 12. Coupling 22, when engaged, prevents hub 12 from rotating relative to probe 21. Preferably coupling 22 holds hub 12 onto the end of probe 21 such that hub 12 will remain coupled to probe 21 even if probe 21 is retracted slightly when positioning hub 12.

In the illustrated embodiment an end 12A of hub 12 is sharp and serves as a leading end to part the subjects tissues to allow passage of hub 12 to a desired implantation location.

Device 10 optionally includes features that are useful for guiding hub 12 to the desired location and/or determining when hub 12 is in the desired location. For example, hub 12 may include one or more of:
a tiny camera operable to transmit an image of a portion of the tissue surrounding the hub;
an ultrasound transducer or transducers operable to detect the location of hub 12 relative to a tissue interface such as a vessel 15. The ultrasound transducer(s) may optionally be ‘receive only’ and used to detect ultrasound generated by an external transmitter and echoes of those externally generated signals from vessel 15. This advantageously avoids the need to incorporate an ultrasound transmitter in device 10.
an infrared source and detector (e.g. a near infrared spectrometry NIRS sensor) operable to detect blood in vessel 15;
an ultrasound reflector which increases the echo signal from hub 12 to facilitate detection of hub 12 by an external ultrasound imaging system;
an antenna connected to an oscillator or transmitter that can be operated to drive the antenna to emit electromagnet radiation that can be picked up from outside of the subject to determine a position of hub 12.

Where hub 12 includes sensors used to determine a position of hub 12 relative to a vessel 15, signals from those sensors may be carried over a wireless data communication system as described above and/or carried by electrical or optical signal carriers included in probe 21.

When hub 12 is at a location at which it is desired to implant hub 12, coupling 22 may be released to allow probe 21 to be withdrawn leaving hub 12 in place in the subject.

Coupling 22 may, for example, comprise a socket carried by one of hub 12 and probe 21 and a projection carried by the other one of hub 12 and probe 21. The socket receives the projection and prevents rotation of the projection relative to the socket. The projection and socket may have any of a wide range of configurations such as square, triangular, splined, oval, rectangular or the like.

Preferably hub 12 fits onto probe 21 in only one orientation and tool 20 comprises markings and/or a handle and/or a configuration such that the orientation of hub 12, when engaged to probe 21 by coupling 22, is known from the orientation of the markings, handle and/or configuration of tool 20 even when hub 12 has entered the patient's skin and is not visible to the eye.

Coupling 22 may optionally grip hub 12 so that hub 12 remains engaged to probe 21 until hub 12 is in a desired position for implantation adjacent to a vessel 15. Grip may be provided in various ways including, without limitation:
by friction (the frictional force may be large enough to pull hub 12 back with arms 14 non extended but small enough that hub 12 will come off of probe 21 when arms 14 are extended and probe 21 is withdrawn);
a mechanically operated mechanism such as a detent or clamp or gripper or sutures that holds hub 12 onto probe 21 and can be released to allow hub 12 to separate from probe 21;
an electrically operated mechanism such as a detent or clamp or gripper or sutures that holds hub 12 onto probe 21 and can be released to allow hub 12 to separate from probe 21.

Arms 14 are preferably initially stored in a compact configuration within a body of hub 12. This facilitates insertion of hub 12 into the subject's body. For example, arms 14 may be rolled up or curled up inside hub 12 or arms 14 may extend side-by side or overlapping across hub 12 in a transverse direction. When hub 12 is in a desired position arms 14 may be extended.

Arms 14 preferably have rounded tips and edges so that they can bluntly pass through loose tissue. Arms 14 may be coated with a slippery layer such as a slippery coating bonded to surfaces of arms 14 and/or a lubricant coating arms 14. For example, arms 14 may comprise rounded tipped cylinders formed to have a desired curvature.

In some embodiments arms 14 extend with a motion that is directed parallel to a long axis of the arms (e.g. tips of the arms are pushed through tissues as the arms 14 are extended). In some embodiments arms 14 extend along arcuate paths.

In some embodiments tool 20 includes an actuating mechanism operable to remotely extend arms 14. The mechanism for remotely extending arms 14 may, for example comprise:
a wire or rod that can be pulled, pushed and/or rotated to extend arms 14 The wire may, for example, extend along a bore of probe 21. Tool 20 may include a knob or handle operable to move the wire or rod to extend arms 14;
a tube carrying fluid that can be pressurized or depressurized to extend arms 14. The tube may, for example, comprise a lumen of probe 21. The fluid may, for example, be a sterile saline solution.
a drive member 40 that can be operated (e.g. turned) by an actuation tool to operate the mechanism to extend or retract arms 14.

In some embodiments the actuating mechanism is operable to extend arms 14 and also to uncouple hub 12 from probe 21. For example, hub 12 may be uncoupled from probe 21 when arms 14 are fully extended, for example by:
extending arms 14 by pulling on a wire and then uncoupling hub 12 by pulling more on the wire;
extending arms 14 by rotating a wire and then uncoupling hub 12 by pulling on the wire;
extending arms 14 by pressurizing a fluid and then releasing hub 12 by increasing a pressure of the fluid and/or delivering more of the pressurized fluid;
extending arms 14 by pushing, pulling or rotating a wire and then uncoupling hub 12 by delivering a pressurized fluid to coupling 22. The pressurized fluid may, for example, be delivered through a lumen surrounding the wire.

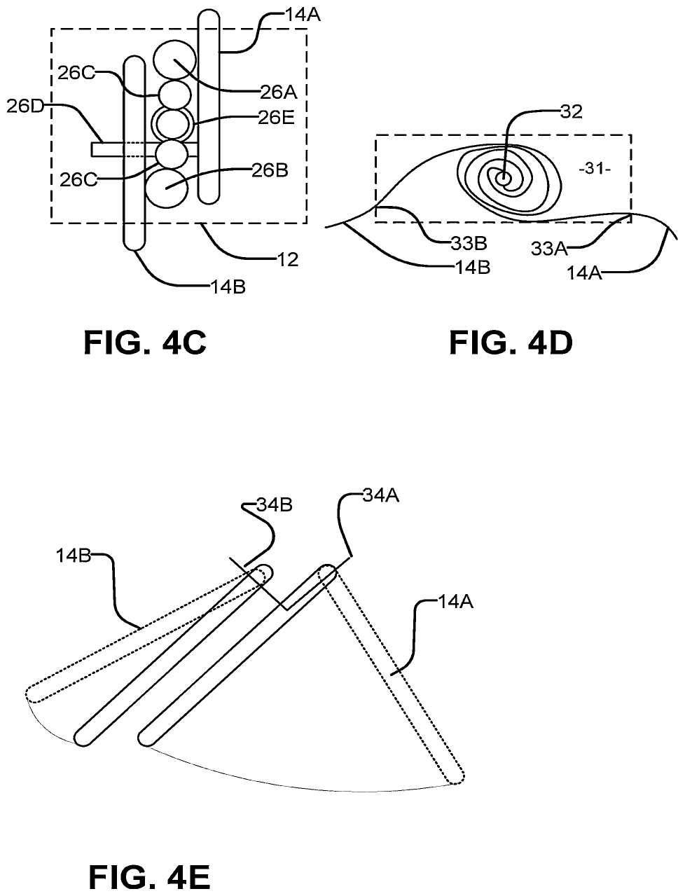

FIGS. 4A, 4B and 4C show some example ways to provide extendible arms 14. Arms 14 are initially contained within a cavity in hub 12. Arms 14 may optionally be made of a flexible material that has a shape memory so that arms 14 tend to take a particular shape (e.g. a curved shape as illustrated schematically in FIG. 2) when extended from hub 12.

FIG. 4A illustrates an example embodiment in which arms 14A and 14B are initially oriented transversely within a body of a hub 12. Arms 14A and 14B are moved transversely in opposite directions so that their tips are pushed out of hub 12 through corresponding openings 37A and 37B. Arms 14A and 14B may, for example, be actuated by a gear train that includes a gear connected to drive extension of each arm 14A and 14B.

FIG. 4B schematically illustrates one example gear train. Gear 25A engages teeth on edges of both of arms 14A and 14B. Rotation of gear 25A causes arms 14A and 14B to move in opposite directions so that arms 14A and 14B may be simultaneously extended or retracted. Gear 25A may be driven to rotate in any suitable manner. In FIG. 4B gear 25A is driven by a drive comprising a worm 25B and wheel 25C. Worm 25B may, for example, be rotated by engagement with a rotatable wire or rod of tool 20. Worm 25B drives wheel 25C to rotate. Gear 25A is coupled to rotate with wheel 25C. The worm and wheel may hold arms 14A and 14B in their extended position.

In another example embodiment gear 25A is driven by engagement with a tool (e.g. a micro screwdriver) that directly drives gear 25A. In another example embodiment gear 25A is turned by a ratchet mechanism that may, for example, be operated by repeatedly pushing a rod or wire of tool 20.

FIG. 4C illustrates another example gear train in which first and second gears or rollers 26A and 26B engage teeth on arms 14A and 14B respectively. Gears 26A and 26B are coupled to turn in the same direction, for example by one or more gears 26C. Gears 26A and 26B are offset to either side of a longitudinal centerline of hub 12 to provide extended travel of arms 14.

Gears 26A, 26B, 26C may be turned to extend or retract arms 14A and 14B in any suitable way. For example, in FIG. 4C, gears 26A, 26B and 26C are driven by a worm and wheel arrangement comprising worm 26D and wheel 26E similar to that described above.

In the example of FIG. 4D, arms 14A and 14B are initially in a cavity 31 within hub 12 and are rolled around a spindle 32. Arms 14A and 14B can be extended to project from hub 12 by turning spindle 32. This causes the tips of arms 14A and 14B to leave cavity 31 by way of slits 33A and 33B. Spindle 32 may be turned until arms 14A and 14B are fully extended (as illustrated schematically in dotted outline). In some embodiments, an angle θ formed between slits 33A and 33B relative to the center of spindle 32 is less than 180 degrees. In such cases arms 14A and 14B may form a dihedral angle that receives a vessel 15.

FIG. 4E illustrates another example embodiment wherein arms 14A and 14B are pivotally mounted to hub 14. Arms 14A and 14B may be pivotal about axes 34A and 34B that extend transversely to a longitudinal centerline of hub 12. Axes 34A and 34B may be oriented at an angle relative to one another such that, when extended, arms 14A and 14B form a dihedral angle in which a vessel 15 may be received.

In any described embodiment, strain gauges and/or pressure sensors may be provided on arms 14A and/or 14B and/or force sensors may be provided to measure forces between arms 14 and/or between one or both of arms 14 and a body of hub 12.

In any embodiment, electrical conductors may carry signals from the strain gauges and/or pressure sensors and/or force sensors to circuitry that receives the signals and transmits the signals or a result of processing the signals to a monitoring device. The strain gauges, pressure sensors and/or electrical conductors may be microfabricated on arms 14.

The paths taken by the electrical conductors can vary depending on the construction of arms 14 and hub 12 and the locations of sensors serviced by the electrical conductors. For example, in the embodiment of FIG. 4D electrical conductors optionally comprise electrical contacts (e.g. ring contacts) on spindle 32. The signals may be carried to processing circuitry by way of brushes in electrical contact with the ring contacts.

Arms 14 may be made from any suitable material(s). The materials should be selected to be biocompatible or coated with a biocompatible material. For example, arms 14 may be made from a material such as a polyamide, a composite including carbon fiber and/or carbon nanotubes etc.

Some example coatings that may be applied to arms 14 and/or any other components of device 10 are described in Fang, Hui et al. Ultrathin, transferred layers of thermally grown silicon dioxide as biofluid barriers for biointegrated flexible electronic systems, PNAS Oct. 18, 2016 113 (42) 11682-11687; https://doi.org/10.1073/pnas.1605269113 which is hereby incorporated herein by reference.

Arms 14 are preferably stiff enough to push their way through tissue as they are being extended from hub 12 without being unduly bent or deflected.

Arms 14 may optionally have surfaces that are smooth and slippery relative to tissues. The slipperiness may be provided by a bio-compatible coating on arms 14, a material of which arms 14 are made and/or a biocompatible gel that is initially present on arms 14.

A system as described herein may process sensor signals to yield instantaneous blood pressure measurements. The instantaneous blood pressure measurements may be processed to determine systolic blood pressure (SBP) and diastolic blood pressure (DBP). The systolic and diastolic pressures may be processed together to yield mean arterial pressure (MAP). MAP may be computed, for example, by MAP=(1/3 SBP)+(2/3 DBP).

An implanted blood pressure monitor as described herein may carry additional sensors. The additional sensors may be useful to correlate/verify blood pressure readings obtained as described elsewhere herein and/or may monitor other functions. For example, an apparatus as described herein may incorporate one or more of:
near infrared spectroscopy (NIRS) sensors. Such sensors may, for example, be located on a face of hub 12 facing toward vessel 15. NIRS sensors may, for example be applied to measure oxygenation of blood in vessel 15.
ultrasound sensors. Such sensors may, for example acquire Doppler ultrasound measurements of blood flow in vessel 15.
one or more blood chemistry sensors. Such sensors may operate with or without direct contact with blood. A blood chemistry sensor that operates when in contact with blood may, for example, project from hub 12 into the lumen of vessel 15 and measure concentrations of one or more biochemicals. In some embodiments activation of a mechanism to extend arms 14 also extends a member carrying the blood chemistry sensors into the lumen of vessel 15. A blood chemistry sensor that does not require contact with blood may, for example, operate by measuring the interaction of blood with light. A blood chemistry sensor may monitor levels of chemical species such as glucose, sodium (Na+), potassium (K+), chloride (Cl—), bicarbonate (HCO3-), urea (e.g. blood urea nitrogen), and/or creatinine.

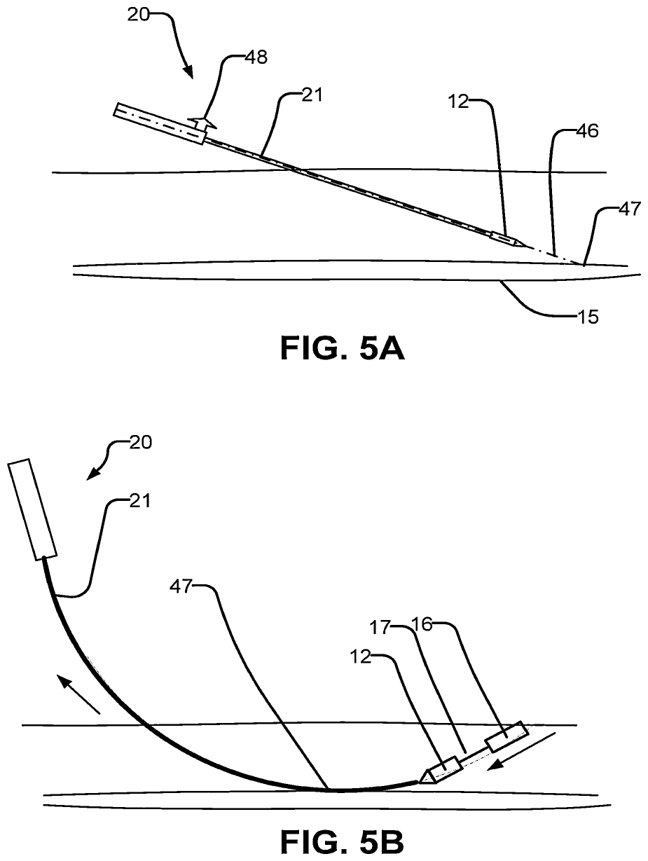

FIGS. 5A and 5B schematically illustrate a method for implanting a blood pressure monitor (for example having a construction as described herein) or another device at a location close to a vessel 15.

In FIG. 5A tool 20 is oriented to be aligned with a path 46 that intersects a vessel 15 at a shallow angle. It is desired to position hub 12 on path 46 at target location 47 adjacent to vessel 15 without penetrating vessel 15.

When hub 12 is delivered to target location 47 (see FIG. 5B) it is intended that hub 12 will be oriented so that arms 14A and 14B can be extended symmetrically on either side of vessel 15.

A user may push tool 20 to drive hub 12 through a subject's tissues along path 46. Tool 20 may optionally be used with a fixture which helps to keep probe 21 of tool 20 aligned with path 46. Tool 20 may optionally be used with an ultrasound system that monitors progress of hub 12 along path 46.

In the illustrated embodiment tool 20 includes a marker 48 that indicates the angular orientation of the side of hub 12 that is intended to face away from vessel 15. A user can adjust the angle of hub 12 as hub 12 is inserted with reference to marker 48.

Once hub 12 is at target location 47, the user may operate tool 20 to extend arms 14A and 14B, for example as described above. The user may also operate tool 20 to disconnect hub 12 from probe 21. These steps may be separate or combined into a single action.

In some embodiments an implanted blood pressure monitor includes hub 12 and a connected module 16 that incorporates batteries, electrical circuits, one or more antennas or other functionality. Module 16 may be implanted together with hub 12 in various ways.

In one example embodiment module 16 is pushed in front of hub 12. Module 16 may have a sharpened tip to facilitate being pushed along path 46. Module 16 may be pushed to a location 47A that is spaced apart from target location 47 and then tool 20 may be retracted to separate hub 12 from module 16 and to bring hub 12 to location 47. Hub 12 and module 16 may remain connected by way of a cable comprising one or more electrical and/or optical conductors.

In another example embodiment probe 21 comprises a slot that receives module 16. Module 16 is carried into the subject's tissues together with hub 12 as tool 20 is advanced along path 46. When tool 20 is detached from hub 12 and retracted module 16 may leave probe 21 and remain implanted in the subject's tissue.

In another example embodiment as illustrated in FIG. 5B, probe 21 is gently curved and is inserted into the subject's tissue so that the deepest point of the curve passes through target location 47. Probe 21 may be advanced until it breaks the subject's skin at a second location. Hub 12 or another device to be implanted near the blood vessel may then be attached at the end of probe 21 and probe 21 may subsequently be retracted to pull hub 12 and connected module 16 back along the curved path following probe 21 until hub 12 is at target location 47. Tool 20 may then be operated to advance arms 14A and 14B and to release probe 21 from hub 12. Probe 21 may then be retracted.

In some embodiments an implanted blood pressure monitor as described herein may be tested to verify that it is picking up a signal that can be processed to yield blood pressure measurements with an adequate signal to noise ratio (SNR) before removing tool 20. For example, outputs of sensors that directly or indirectly measure changing forces on arms 14 as a result of pulsations of vessel 15 may be processed to measure a SNR of the signals. In such cases a physician or other trained medical person may adjust the position and/or orientation of hub 12 and/or adjust the extension of arms 14 in an attempt to improve the quality (e.g. by increasing SNR) of the detected signals.

After it has been implanted a blood pressure measurement system as described herein may be calibrated by comparing signals output by sensors which measure forces on arms 14 directly or indirectly with blood pressure measurements made by another reliable system such as a intra-arterial catheter, measurement by a trained person using a sphygmomanometer and stethoscope, an automated blood pressure measurement device that applies cuff occlusion with an ausculatory or oscillometric algorithm to estimate blood pressure, or a system that uses pulse wave velocity to estimate blood pressure.

Calibration may involve, for example, fitting parameters to an equation that relates output values S for sensor(s) on hub 10 and/or time derivatives of output values S to blood pressure values. The equation may be a linear equation, a polynomial equation, another non-linear equation etc. In some embodiments the calibration is stored in the form of values for calibration parameters (e.g. parameters A and B in the equation “A*S+B” or parameters A, B and C in the equation “A*S²+B*S+C”. In some embodiments the calibration is stored in the form of a lookup table which uses values for S and/or time derivative(s) of S as keys to look up a blood pressure value.

In some embodiments a trained machine learning system is applied to periodically or continuously update the calibration of a device 10 to maintain accuracy over extended periods. The machine learning system may be trained based on a number of implanted devices 10 for which blood pressure was also monitored using another reliable modality. The machine learning system may be trained to take as inputs:
outputs from sensor(s) of device 10;
a blood pressure determined by processing those outputs according to a current calibration; and/or
parameters of a current calibration; and/or
blood pressure readings for the subject obtained by another modality;
   to provide revised calibration parameter values.

In some embodiments the machine learning system comprises a random forest regression or a feed forward neural network.

Some embodiments provide a mechanism to extend arms 14 that is activated by an activation tool 41 (e.g. a micro screwdriver) that is engaged to a drive fitting 40 (e.g. a head that is slotted or has a recessed socket or projecting shape configured to engage the activation tool) on hub 12. Activation tool 41 may be engaged to drive fitting 40 after hub 12 has been implanted. Activation tool 41 may be operated to extend arms 14.

In embodiments where device 10 has a drive fitting 40 designed to receive a tool 41 after device 10 is implanted in a subject (e.g. to extend or retract arms 14), device 10 may include features which assist in guiding the tool 41 to the drive fitting 40 and/or retaining the tool 41 on the drive fitting 40 while drive fitting 40 is operated. These features may include, for example, one or more of:
a guide such as a funnel shape arranged to guide a tip of tool 41 into engagement with a drive fitting 40; and/or
a beacon such as an ultrasound beacon, an electromagnetic beacon or the like that emits a signal that can be detected by a sensor on tool 41 to assist in guiding tool 41 to the drive fitting. The received signal may, for example change based on the distance from tool 41 to the drive fitting and/or the orientation of tool 41 relative to the drive fitting.

An implanted blood pressure monitor as described herein may provide reliable blood pressure measurements for extended periods of time (e.g. days, weeks, months or years).

An implantable device of the type described herein may be removed in various ways. For example, an incision may be made to expose hub 12 (and module 16 if present). Arms 14 may then be retracted (or cut off) and then the implanted device may be removed through the incision. In an example embodiment, hub 12 includes a drive fitting 40 as described above. Drive fitting 40 may be the same or different from a drive fitting used to extend arms 14. The implanted device may then be prepared for removal by operating the drive fitting 40 to retract arms 14 using an activation tool 41.

After arms 14 are retracted an extraction tool may be used to pull hub 12 (and module 16 if present) out of the patient.

First large face 42A and second large face 42B of hub 12 may be detached from one another, by way of unscrewing, unclipping, disconnecting or the like.

In an example embodiment, tool 41 is configured to couple to a drive fitting 40 that is operable to retract arms 14. Tool 41 may include a torque limiting coupling which allows a user to turn drive fitting 40 with sufficient torque to retract arms 14 but prevents the user from applying too much torque to drive fitting 14. Hub 12 could then be removed by pulling on tool 41. In such embodiments hub 12 may have a very slippery coating such as Teflon so that hub 12 can slide out of the subject.

Many variations are possible in the practice of this invention. For example, apparatus as described herein may include more than two arms. The apparatus may, for example include three arms 14, with two arms 14 extending from one side of hub 12 and one arm 14 extending from the other side of hub 12. In some embodiments the one arm is located longitudinally between the two arms.

### Interpretation of Terms

Unless the context clearly requires otherwise, throughout the description and the claims:
“comprise”, “comprising”, and the like are to be construed in an inclusive sense, as opposed to an exclusive or exhaustive sense; that is to say, in the sense of “including, but not limited to”;
“connected”, “coupled”, or any variant thereof, means any connection or coupling, either direct or indirect, between two or more elements; the coupling or connection between the elements can be physical, logical, or a combination thereof;
“herein”, “above”, “below”, and words of similar import, when used to describe this specification, shall refer to this specification as a whole, and not to any particular portions of this specification;
“or”, in reference to a list of two or more items, covers all of the following interpretations of the word: any of the items in the list, all of the items in the list, and any combination of the items in the list;
the singular forms “a”, “an”, and “the” also include the meaning of any appropriate plural forms.

Words that indicate directions such as “vertical”, “transverse”, “horizontal”, “upward”, “downward”, “forward”, “backward”, “inward”, “outward”, “left”, “right”, “front”, “back”, “top”, “bottom”, “below”, “above”, “under”, and the like, used in this description and any accompanying claims (where present), depend on the specific orientation of the apparatus described and illustrated. The subject matter described herein may assume various alternative orientations. Accordingly, these directional terms are not strictly defined and should not be interpreted narrowly.

Where a component (e.g. an arm, a gear, an assembly, a module, a sensor, a circuit, etc.) is referred to above, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

Specific examples of systems, methods and apparatus have been described herein for purposes of illustration. These are only examples. The technology provided herein can be applied to systems other than the example systems described above. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

Various features are described herein as being present in “some embodiments” or as being “for example”. Such features are not mandatory and may not be present in all embodiments. Embodiments of the invention may include zero, any one or any combination of two or more of such features. This is limited only to the extent that certain ones of such features are incompatible with other ones of such features in the sense that it would be impossible for a person of ordinary skill in the art to construct a practical embodiment that combines such incompatible features. Consequently, the description that “some embodiments” possess feature A and “some embodiments” possess feature B should be interpreted as an express indication that the inventors also contemplate embodiments which combine features A and B (unless the description states otherwise or features A and B are fundamentally incompatible).

It is therefore intended that the following appended claims and claims hereafter introduced are interpreted to include all such modifications, permutations, additions, omissions, and sub-combinations as may reasonably be inferred. The scope of the claims should not be limited by the preferred embodiments set forth in the examples, but should be given the broadest interpretation consistent with the description as a whole.

1. An apparatus for measuring blood pressure comprising:
a first body dimensioned to be positioned with a first face of the first body adjacent to an outer wall of a blood vessel;
at least one element connected to the first body, the at least one element configured and dimensioned to at least partly surround the outer wall of the blood vessel;
one or more sensors operative to detect forces applied on the at least one element and/or displacement of the at least one element relative to the first body resulting from pulsations of the blood vessel; and
a module communicatively connected to the one or more sensors, operative to process signals detected by the one or more sensors and to output a blood pressure measurement.

2. (canceled)

3. The apparatus according to claim 1, wherein the at least one element comprises a first arm and a second arm extending transversely from opposite sides of the first body.

4. (canceled)

5. The apparatus according to claim 3, wherein the first and second arms are displaceable in a radial direction relative to the blood vessel.

6. The apparatus according to claim 3, wherein each of the first and second arms is formed to curve around the blood vessel with a radius of curvature.

7.-8. (canceled)

9. The apparatus according to claim 3, wherein the one or more sensors are arranged on one or both of the first and second arms.

10. The apparatus according to claim 3, wherein the first body comprises a hub and the first and second arms are retractable into a cavity within the hub.

11. The apparatus according to claim 10, comprising first and second openings defined in opposite sides of the hub, the first and second arms being extendable through the first and second openings respectively from within the cavity.

12. The apparatus according to claim 11, further comprising an actuation system arranged within the hub, the actuation system operatively connected to the first and/or second arms and operable to move the first and second arms between a retracted position and an extended position.

13. The apparatus according to claim 12, wherein the actuation mechanism comprises a rotatable spindle arranged within the hub wherein the first and second arms are wound around the spindle when the first and second arms are in the retracted position.

14. (canceled)

15. The apparatus according to claim 13, wherein the first and second openings comprise first and second slits and an angle between the first and second slits relative to a center of the spindle is less than 180 degrees.

16. The apparatus according to claim 3, wherein the first and second arms are pivotally mounted to the first body.

17.-20. (canceled)

21. The apparatus according to claim 3, wherein the first arm and/or the second arm is made of a flexible material.

22. (canceled)

23. The apparatus according to claim 1, wherein the at least one element comprises a second body and, the sensors are connectable between the first body and the second body.

24.-27. (canceled)

28. The apparatus according to claim 1, wherein the one or more sensors are selected from the group consisting of: strain gauges, pressure sensors, force sensors, distance sensors, angular position sensors, near infrared spectroscopy (NIRS) sensors, ultrasound sensors, and blood chemistry sensors.

29. (canceled)

30. The apparatus according to claim 1, wherein the first face of the first body comprises a concave surface.

31. (canceled)

32. The apparatus according claim 30, wherein the first face of the first body subtends an arc in the range of from about 50 degrees to about 60 degrees around a center of curvature of the first face.

33.-35. (canceled)

36. The apparatus according to claim 1, further comprising:
an introducer tool;
a probe extending from the introducer tool; and
a coupling releasably connecting an end of the probe to the first body.

37. The apparatus according to claim 36, wherein the introducer tool comprises an actuating mechanism operative to remotely control a movement of the first and second arms between the extended position and the retracted position.

38.-40. (canceled)

41. The apparatus according to claim 1, wherein the first body comprises a sharp distal end along a longitudinal axis of the first body.

42. The apparatus according to claim 1, wherein the module is communicatively connected to a receiver, and the module is operative to transmit the blood pressure measurement to the receiver.

43.-80. (canceled)

81. The apparatus according to claim 1 wherein the at least one element comprises a second body dimensioned to be positioned with a first face of the second body adjacent to the outer wall of the blood vessel.

82. The apparatus according to claim 81 wherein the first body and the second body are connected by extensible elements configured to allow the first body and the second body to move toward and away from one another.

83. The apparatus according to claim 81 wherein the one or more sensors are operative to measure:
forces applied on and/or between the first body and/or the second body; and/or
displacement of the first body and the second body relative to one another;
which occur as a result of changing pressure within the blood vessel.
